(19) United States
(12) Patent Application Publication
(10) Pub. No.: US 2006/0073106 A1
(43) Pub. Date: Apr. 6, 2006

(54) Novel stabilized cinnamic ester sunscreen compositions

(75) Inventors: Katja Berg-Schultz, Kaiseraugst (CH); Volker Schehlmann, Schopfheim (DE); Horst Westenfelder, Neustadt a d W (DE)

Correspondence Address: NIXON & VANDERHYE, PC, 901 NORTH GLEBE ROAD, 11TH FLOOR, ARLINGTON, VA, 22203, US

(73) Assignee: DSM IP Assets B.V., TE Heerlen (NL)

(21) Appl. No.: 10/542,927

(22) PCT Filed: Sep. 25, 2003
(86) PCT No.: PCT/EP03/10685

§ 371 Date: Jul. 21, 2005

(30) Foreign Application Priority Data
Feb. 3, 2003 (EP) 03002290.9

(51) Int. Cl. A61K 8/37

(52) U.S. Cl. 424/59

(57) ABSTRACT

The invention relates to a method of enhancing the photostability of an encapsulated cinnamate derivative in a topical sunscreen composition which comprises introducing into such sunscreen composition an effective amount of at least one additional non-encapsulated sunscreen.

The present invention relates to a photostable cosmetic or pharmaceutical light screening composition containing an encapsulated cinnamic ester and at least one additional non-encapsulated UV-A and/or UV-B and/or a broad spectrum sunscreen for shielding the skin from ultraviolet radiation. Furthermore the invention relates to a method for enhancing the photostability of encapsulated cinnamic ester derivatives by adding at least one additional non-encapsulated sunscreen. The preferred compositions and methods of the present invention use a microencapsulated 2-ethylhexyl-4-methoxy cinnamate (EHMC) sunscreen, an additional non-encapsulated sunscreen, and a cosmetically acceptable vehicle. The additional sunscreen can be selected from UV-A and/or UV-B and/or broad spectrum sunscreens, or, preferably, from a combination thereof.

Cinnamate derivatives such as ethylhexyl methoxycinnamate are known to be useful as sunscreen agents, particularly for protecting human skin, e.g. in cosmetic formulations. To prevent unwanted effects, e.g., allergic reactions of such sunscreen agents, or to avoid their cross reactivity with other sunscreen active ingredients (e.g. butyl methoxydibenzoylmethane) it has been proposed to encapsulate them in a various coating matrix which may e.g. be silica or organically modified silica (see International applications WO 00/09652, WO 00/71084 and WO 00/72806). Surprisingly, it has been found that such encapsulated cinnamate derivatives itself undergo photodecomposition during irradiation resulting in a significant loss of absorbance, and, thus, photoprotection.

In accordance with the present invention it has surprisingly been found that the photostability of encapsulated cinnamic ester derivatives (in the following referred to as ‘encapsulated cinnamates’) in a formulation for application on the human skin can be improved by adding an effective amount of at least one additional non-encapsulated sunscreen to such formulations.

Accordingly, the present invention relates to a method of enhancing the photostability of encapsulated cinnamates in topical sunscreen compositions by introducing in such sunscreen composition an effective amount of at least one additional non-encapsulated sunscreen, preferably a UV-B or a broad spectrum sunscreen or a combination of UV-B and/or UV-A and/or broad spectrum sunscreens.

The term “encapsulated cinnamates” refers to a UV absorbing cinnamic acid derivative being either a discrete liquid or solid particles which are coated by a suitable capsule wall material to form microcapsules of a core-shell type. These microcapsules may be prepared by various polymerization techniques known in the art such as a sol-gel method, a solvent evaporation method, a coacervation method, an interfacial polymerization method or an emulsion/interfacial emulsion polymerization method. Such capsules containing the UV absorbing cinnamic acid derivative as core material typically have a mean diameter of about 0.01 μm to about 100 μm. Of particular interest are capsules having a diameter of 0.1-10 μm.

The coating may be formed of any polymer conventionally used such as e.g. polyacrylate, polyurethans, polyamides or silicon based polymers. Of particular interest are microcapsules having as core a cinnamic acid derivative which is surrounded by a shell of silicon based polymer such as ‘sol-gel glass’, a silicon-based network polymer or a ‘silicone-resin polypeptide’ as disclosed, e.g., in WO 00/72806 and EP 934773, respectively.

For the purposes of the present invention the cinnamic ester derivatives are preferably of the general formula I 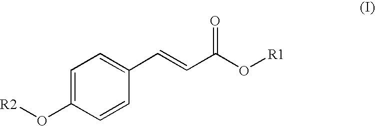
wherein R¹, R², are) independently, hydrogen or saturated straight or branched chain alkyl containing 1 to 21, preferably 1 to 8 carbon atoms, such as methyl, ethyl, propyl, isopropyl, butyl, sec. butyl, isobutyl, pentyl, neopentyl, hexyl and 2-ethyl-hexyl.

Cinnamate derivatives of forumla I of particular interest are 2-ethyl-hexyl methoxycinnamate (PARSOL® MCX), ethoxyethyl methoxycinnamate, and isoamyl methoxycinnamate.

The ‘encapsulated cinnamates’ for use in the present invention may be prepared by means of various effective encapsulation technologies and encapsulation materials. Suitable microencapsulation can be obtained, without being limited thereto, via a sol-gel method (e.g. described in WO 00/72806), a solvent evaporation method (e.g. described in European Polymer Journal (2001), 37(5), 955-963, J. Controlled release 13, 33-41), a coacervation method (e.g. described in WO 9822210), an interfacial polymerization method (e.g. described in DE 2722973), a solvent evaporation method, an emulsion/interfacial emulsion polymerisation or according to a method described e.g. in EP 0934773 and in Fragrance Journal 2002, (30)7, 62-67. Such encapsulation techniques are familiar to the person skilled in the art. The ratio of capsule material to cinnamate derivative such as EHMC can vary be between 1-99% preferable between 10-90%. The final product can be an aqueous dispersion of varying payload and volume fraction as well as a dried powder.

In the sol-gel method preferably sol-gel silica, (see International applications WO 00/09652 and WO 00/72806), the cinnamate derivative is dissolved in the sol-gel precursors wherein the sol-gel precursors can be a metal or a semi metal alkoxide monomer, or a partially hydrolyzed and partially condensed polymer thereof, or a mixture of any of the above. This solution is emulsified under high shear forces in an aqueous solution, containing surfactants, such as cetyltrimethylammonium chloride and the like and/or protective colloids such as PVP (polyvinylpyrrolidone), PVA (polyvinylalcohol) and the like, that assist in stabilizing the emulsions. The obtained emulsion is mixed with an aqueous solution at a suitable selected pH (basic, neutral or acidic), until spheres containing the encapsulated cinnamate derivatives are formed.

In the solvent evaporation method, the sunscreen active ingredient is dissolved in a volatile solvent, which is insoluble in water. In the same solution, a polymer such as polylactic acid is dissolved. Thereafter, the solution is added to an aqueous solution, which contains an emulsifier such as ethoxylated sorbitan monolaureate (Tween® 20, ICI), sorbitan oleate, sorbitan sesquioleate, sorbitan isostearate, sorbitan trioleate, polyglyceryl-3-diisostearate, polyglycerol esters of oleic/isostearic acid, polyglyceryl-6 hexaricinolate, polyglyceryl-4-oleate, polygylceryl-4 oleate/PEG-8 propylene glycol cocoate, oleamide DEA, TEA myristate, TEA stearate, magnesium stearate, sodium stearate, potassium laurate, potassium ricinoleate, sodium cocoate, sodium tallowate, potassium castorate, sodium oleate, and mixtures thereof. Further suitable emulsifiers are phosphate esters and the salts thereof such as cetyl phosphate (Amphisol® A), diethanolamine cetyl phosphate (Amphisol®), potassium cetyl phosphate (Amphisol® K), sodium glyceryl oleate phosphate, hydrogenated vegetable glycerides phosphate and mixtures thereof. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof. After high shear mixing, an emulsion is formed. The solvent is removed by evaporation under reduced pressure resulting in the formation of microspheres, which contain the cinnamate sunscreening agent entrapped within the polymer matrix.

In the coacervation method, the cinnamate derivative is dissolved in a non volatile solvent (i.e. soybean oil, mineral oil and the like), or used as if liquid, and emulsified in water which contains a water soluble polymer such as gelatin, gliadin and the like. After an emulsion with proper particle size is formed, a coacervation agent, such as Na₂SO₄, MgSO₄ and the like is added, leading to coacervation of the water soluble polymer around each droplet. Thereafter, a suitable crosslinking agent is added thus forming a rigid wall around the oil droplet.

In the interfacial polymerization method, a suitable monomer or a monomer mixture is dissolved in the cinnamate derivative (if liquid), or in a solution containing the cinamate derivative, and then emulsified in an aqueous solution which contains a suitable emulsifier. After the emulsion is formed, a second monomer or monomer mixture, which is water soluble, is added to the emulsion. The polymerization occurs at the oil water interface of the droplets, resulting in the formation of a wall. The monomers can be chosen so as to promote a variety of interfacial polymerization products as wall materials such as polyamides, polyesters, polyureas or mixed condensation products like polyesterurethanes, polyesteramides and the like.

In the emulsion polymerization/interfacial emulsion polymerization the cinnamate derivative (if liquid), or a solution containing the cinnamate derivative, is emulsified in an aqueous solution which contains a suitable emulsifier (e.g. cetyl trimethyl ammonium chlorid). After the emulsion is formed, a suitable monomer or a monomer mixture is added to the emulsion. The addition of a suitable catalyst is not obligatory but if necessary it can be added e.g. to the cinnamate derivative or after the addition of the monomer. The polymerization occurs at the oil water interface of the droplets, resulting in the formation of a wall.

The preparation of said topical sunscreen compositions is well known to the skilled artisan in this field. For the preparation of said topical sunscreen compositions, especially preparations for dermatological and/or cosmetic use, such as skin protection and sunscreen formulations for everyday cosmetics an ‘encapsulated cinnamate’ and at least one additional non-encapsulated sunscreen active agent can be incorporated in auxiliary agents, e.g. a cosmetic base, which are conventionally used for such formulations. Where convenient, other conventional UV-A and/or UV-B and/or broad spectrum screening agents may also be added. The combination of UV screens may show a synergistic effect. The amount of the ‘encapsulated cinnamate’ and other known UV-screens is not very critical. Suitable amounts of the ‘encapsulated cinnamate’ are about 10 to about 50% by weight (depending on the payload and volume fraction of the microcapsules) and about 0.5-12% by weight of at least one additional, hydrophilic and/or lipophilic UV-A or UV-B or broad spectrum screening agent. These additional screening agents are advantageously selected from among the compounds listed below without being limited thereto:

Examples of UV B or broad spectrum screening agents, i.e. substances having absorption maxima between about 290 and 340 nm, which come into consideration for combination with the compounds of the present invention are for example the following organic and inorganic compounds:
Acrylates such as 2-ethylhexyl 2-cyano-3,3-diphenylacrylate (octocrylene, PARSOL® 340), ethyl 2-cyano-3,3-diphenylacrylate and the like;
Camphor derivatives such as 4-methyl benzylidene camphor (PARSOL® 5000), 3-benzylidene camphor, camphor benzalkonium methosulfate, polyacrylamidomethyl benzylidene camphor, sulfo benzylidene camphor, sulphomethyl benzylidene camphor, therephthalidene dicamphor sulfonic acid and the like;
Cinnamate derivatives such as octyl methoxycinnamate (PARSOL® MCX), ethoxyethyl methoxycinnamate, diethanolamine methoxycinnamate (PARSOL® Hydro), isoamyl methoxycinnamate and the like as well as cinnamic acid derivatives bond to siloxanes;
p-aminobenzoic acid derivatives, such as p-aminobenzoic acid, 2-ethylhexyl p-dimethylaminobenzoate, N-oxypropylenated ethyl p-aminobenzoate, glyceryl p-aminobenzoate,
Benzophenones such as benzophenone-3, benzophenone-4,2,2′,4,4′-tetrahydroxy-benzophenone, 2,2′-dihydroxy-4,4′-dimethoxybenzophenone and the like;
Esters of Benzalmalonic acid such as di-(2-ethylhexyl) 4-methoxybenzalmalonate
Esters of 2-(4-ethoxy-anilinomethylene)propandioic acid such as 2-(4-ethoxy anilinomethylene)propandioic acid diethyl ester as described in the European Patent Publication EP 0895 776
Organosiloxane compounds containing benzmalonate groups as described in the European Patent Publications EP 0358584 B1, EP 0538431 B1 and EP 0709080 A1;
Drometrizole trisiloxane (Mexoryl XL)
Pigments such as microparticulated TiO₂, and the like. The term “microparticulated” refers to a particle size from about 5 nm to about 200 nm, particularly from about 15 nm to about 100 nm. The TiO₂ particles may also be coated by metal oxides such as e.g. aluminum or zirconium oxides or by organic coatings such as e.g. polyols, methicone, aluminum stearate, alkyl silane. Such coatings are well known in the art.
Imidazole derivatives such as e.g. 2-phenyl benzimidazole sulfonic acid and its salts (PARSOL®HS). Salts of 2-phenyl benzimidazole sulfonic acid are e.g. alkali salts such as sodium- or potassium salts, ammonium salts, morpholine salts, salts of primary, sec. and tert. amines like monoethanolamine salts, diethanolamine salts and the like.
Salicylate derivatives such as isopropylbenzyl salicylate, benzyl salicylate, butyl salicylate, octyl salicylate (NEO HELIOPAN OS), isooctyl salicylate or homomenthyl salicylate (homosalate, HELIOPAN) and the like;
Triazine derivatives such as octyl triazone (UVINUL T-150), dioctyl butamido triazone (UVASORB HEB), bis ethoxyphenol methoxyphenyl triazine (Tinosorb S) and the like.

Examples of broad spectrum or UV A screening agents i.e. substances having absorption maxima between about 320 and 400 nm, which come into consideration for combination with the compounds of the present invention are for example the following organic and inorganic compounds:
Dibenzoylmethane derivatives such as 4-tert. butyl-4′-methoxydibenzoyl-methane (PARSOL® 1789), dimethoxydibenzoylmethane, isopropyldibenzoylmethane and the like;
Benzotriazole derivatives such as 2,2′-methylene-bis-(6-(2H-benzotriazole-2-yl)-4-(1,1,3,3,-tetramethylbutyl)-phenol (TINOSORB M) and the like;
phenylene-1,4-bis-benzimidazolsulfonic acids or salts such as 2,2-(1,4-phenylene)bis-(1H-benzimidazol-4,6-disulfonic acid) (Neoheliopan AP)
amino substituted hydroxybenzophenones such as 2-(4-Diethylamino-2-hydroxy-benzoyl)-benzoic acid hexylester as described in the European Patent Publication EP 1046391
Pigments such as microparticulated ZnO or TiO₂ and the like. The term “microparticulated” refers to a particle size from about 5 nm to about 200 nm, particularly from about 15 nm to about 100 nm. The particles may also be coated by other metal oxides such as e.g. aluminum or zirconium oxides or by organic coatings such as e.g. polyols, methicone, aluminum stearate, alkyl silane. Such coatings are well known in the art.

As dibenzoylmethane derivatives have limited photostability it may be desirable to photostabilize these UV-A screening agents. Thus, the term “conventional UV-A screening agent” also refers to dibenzoylmethane derivatives such as e.g. PARSOL® 1789 stabilized by, e.g.,
3,3-Diphenylacrylate derivatives as described in the European Patent Publications EP 0 514 491 B1 and EP 0 780 119 A1;
Benzylidene camphor derivatives as described in the U.S. Pat. No. 5,605,680;
Organosiloxanes containing benzmalonate groups as described in the European Patent Publications EP 0358584 B1, EP 0538431 B1 and EP 0709080 A1.

The compositions of the invention can also contain usual cosmetic adjuvants and additives, such as preservatives/antioxidants, fatty substances/oils, water, organic solvents, silicones, thickeners, softeners, emulsifiers, additional sunscreens, antifoaming agents, moisturizers, fragrances, surfactants, fillers, sequestering agents, anionic, cationic, nonionic or amphoteric polymers or mixtures thereof, propellants, acidifying or basifying agents, dyes, colorants, pigments or nanopigments, in particular those suited for providing an additional photoprotective effect by physically blocking out ultraviolet radiation, or any other ingredients usually formulated into cosmetics, in particular for the production of sunscreen/antisun compositions. The necessary amounts of the cosmetic and dermatological adjuvants and additives can, based on the desired product, easily be chosen by a skilled artisan in this field and will be illustrated in the examples, without being limited hereto.

An additional amount of antioxidants/preservatives is generally preferred. Based on the invention all known antioxidants usually formulated into cosmetics can be used. Especially preferred are antioxidants chosen from the group consisting of amino acids (e.g. glycine, histidine, tyrosine, tryptophane) and their derivatives, imidazole (e.g urocanic acid) and derivatives, peptides such as D,L-carnosine, D-carnosine, L-carnosine and derivatives (e.g. anserine), carotenoids, carotenes (e.g. α-carotene, β-carotene, lycopene) and derivatives, chlorogenic acid and derivatives, lipoic acid and derivatives (e.g. dihydrolipoic acid), aurothioglucose, propylthiouracil and other thiols (e.g. thioredoxine, glutathione, cysteine, cystine, cystamine and its glycosyl-, N-acetyl-, methyl-, ethyl-, propyl-, amyl-, butyl- and lauryl-, palmitoyl-; oleyl-, y-linoleyl-, cholesteryl- and glycerylester) and the salts thereof, dilaurylthiodipropionate, distearylthiodipropionate, thiodipropionic acid and its derivatives (ester, ether, peptides, lipids, nucleotides, nucleosides and salts) as well as sulfoximine compounds (such as buthioninsulfoximine, homocysteinsulfoximine, buthioninsulfone, penta-, hexa-, heptathioninsulfoximine) in very low compatible doses (e.g. pmol bis μmol/kg), additionally (metal)-chelators (such as α-hydroxyfatty acids, palmic-, phytinic acid, lactoferrin), β-hydroxyacids (such as citric acid, lactic acid, malic acid), huminic acid, gallic acid, gallic extracts, bilirubin, biliverdin, EDTA, EGTA and its derivatives, unsaturated fatty acids and their derivatives (such as γ-linoleic acid, linolic acid, oleic acid), folic acid and its derivatives, ubiquinone and ubiquinol and their derivatives, vitamine C and derivatives (such as ascorbylpalmitate and ascorbyltetraisopalmitate, Mg-ascorbylphosphate, Na-ascorbylphosphate, ascorbylacetate), tocopherole and derivates (such as vitamine-E-acetate), mixtures of nat. vitamine E, vitamine A and derivatives (vitamine-A-palmitate and -acetate) as well as coniferylbenzoat, rutinic acid and derivatives, α-glycosylrutin, ferulic acid, furfurylidenglucitol, carnosin, butylhydroxytoluene, butylhydroxyanisole, trihydroxybutyrophenone, urea and its derivatives, mannose and derivatives, zinc and derivatives (e.g. ZnO, ZnSO₄), Selen and derivatives (e.g. selenomethionin), stilbenes and derivatives (such as stilbenoxide, trans-stilbenoxide) and suitable derivatives (salts, esters, ethers, sugars, nucleotides, nucleosides, peptides and lipids) of the named active ingredients. One or more preservatives/antioxidants may be present in an amount about 0.01 wt. % to about 10 wt. % of the total weight of the composition of the present invention. Preferably, one or more preservatives/antioxidants are present in an amount about 0.1 wt. % to about 1 wt. %.

Typically formulations also contain surface active ingredients like emulsifiers, solubilizers and the like. An emulsifier enables two or more immiscible components to be combined homogeneously. Moreover, the emulsifier acts to stabilize the composition. Emulsifiers that may be used in the present invention in order to form O/W, W/O, O/W/O or W/O/W emulsions/microemulsions include sorbitan oleate, sorbitan sesquioleate, sorbitan isostearate, sorbitan trioleate, polyglyceryl-3-diisostearate, polyglycerol esters of oleic/isostearic acid, polyglyceryl-6 hexaricinolate, polyglyceryl-4-oleate, polygylceryl-4 oleate/PEG-8 propylene glycol cocoate, oleamide DEA, TEA myristate, TEA stearate, magnesium stearate, sodium stearate, potassium laurate, potassium ricinoleate, sodium cocoate, sodium tallowate, potassium castorate, sodium oleate, and mixtures thereof Further suitable emulsifiers are phosphate esters and the salts thereof such as cetyl phosphate (Amphisol® A), diethanolamine cetyl phosphate (Amphisol®), potassium cetyl phosphate (Amphisol® K), sodium glyceryl oleate phosphate, hydrogenated vegetable glycerides phosphate and mixtures thereof. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof. The preferred emulsifiers are cetyl phosphate (Amphisol® A), diethanolamine cetyl phosphate (Amphisol®), potassium cetyl phosphate (Amphisole K), PVP Eicosene copolymer, acrylates/C₁₀₋₃₀-alkyl acrylate crosspolymer, PEG-20 sorbitan isostearate, sorbitan isostearate, and mixtures thereof. The one or more emulsifiers are present in a total amount about 0.01 wt. % to about 20 wt. % of the total weight of the composition of the present invention. Preferably, about 0.1 wt. % to about 10 wt. % of emulsifiers are used.

The lipid phase can advantageously be chosen from:
mineral oils and mineral waxes;
oils such as triglycerides of caprinic acid or caprylic acid, preferable castor oil;
oils or waxes and other natural or synthetic oils, in an preferred embodiment esters of fatty acids with alcohols e.g. isopropanol, propyleneglycol, glycerine or esters of fatty alcohols with carbonic acids or fatty acids;
alkylbenzoates; and/or
silicone oils such as dimethylpolysiloxane, diethylpolysiloxane, diphenylpolysiloxane, cyclomethicones and mixtures thereof.

Exemplary fatty substances which can be incorporated in the oil phase of the emulsion, microemulsion, oleo gel, hydrodispersion or lipodispersion of the present invention are advantageously chosen from esters of saturated and/or unsaturated, linear or branched alkyl carboxylic acids with 3 to 30 carbon atoms, and saturated and/or unsaturated, linear and/or branched alcohols with 3 to 30 carbon atoms as well as esters of aromatic carboxylic acids and of saturated and/or unsaturated, linear or branched alcohols of 3-30 carbon atoms. Such esters can advantageously be selected from octylpalmitate, octylcocoate, octylisostearate, octyldodecylmyristate, cetearylisononanoate, isopropylmyristate, isopropylpalmitate, isopropylstearate, isopropyloleate, n-butylstearate, n-hexyllaureate, n-decyloleat, isooctylstearate, isononylstearate, isononylisononanoate, 2-ethyl hexylpalmitate, 2-ethylhexyllaurate, 2-hexyldecylstearate, 2-octyldodecylpalmitate, stearylheptanoate, oleyloleate, oleylerucate, erucyloleate, erucylerucate, tridecylstearate, tridecyltrimellitate, as well as synthetic, half-synthetic or natural mixtures of such esters e.g. jojoba oil.

Other fatty components suitable for use in the formulation of the present invention include polar oils such as lecithines and fatty acid triglycerides, namely triglycerol esters of saturated and/or unsaturated, straight or branched carboxylic acid with 8 to 24 carbon atoms, preferably of 12 to 18 carbon-atoms whereas the fatty acid triglycerides are preferably chosen from synthetic, half synthetic or natural oils (e.g. cocoglyceride, olive oil) sun flower oil, soybean oil, peanut oil, rape seed oil, sweet almond oil, palm oil, coconut oil, castor oil, hydrogenated castor oil, wheat oil, grape seed oil, macadamia nut oil and others); apolar oils such as linear and/or branched hydrocarbons and waxes e.g. mineral oils, vaseline (petrolatum); paraffins, squalan and squalen, polyolefines, hydrogenated polyisobutenes and isohexadecanes, favored polyolefines are polydecenes; dialkyl ethers such as dicaprylylether; linear or cyclic silicone oils such as preferably cyclomethicone (octamethylcyclotetrasiloxane; cetyldimethicone, hexamethylcyclotrisiloxane, polydimethylsiloxane, poly(methylphenylsiloxane) and mixtures thereof.

Other fatty components which can advantageously be incorporated in formulations of the present invention are isoeikosane; neopentylglykoldiheptanoate; propylenglykoldicaprylate/dicaprate; caprylic/capric/diglycerylsuccinate; butylenglykol caprylat/caprat; C₁₂₋₁₃-alkyllactate; di-C₁₂₋₁₃ alkyltartrate; triisostearin; dipentaerythrityl hexacaprylat/hexacaprate; propylenglykolmonoisostearate; tricaprylin; dimethylisosorbid. Especially beneficial is the use of mixtures C₁₂₋₁₅-alkylbenzoate and 2-ethylhexylisostearate, mixtures C₁₂₋₁₅-alkylbenzoate and isotridecylisononanoate as well as mixtures of C₁₂₋₁₅-alkylbenzoate, 2-ethylhexylisostearate and isotridecylisononanoate.

The oily phase of the formulation of the present invention can also contain natural vegetable or animal waxes such as bee wax, china wax, bumblebee wax and other waxes of insects as well as shea butter and cocoa butter.

A moisturizing agent may be incorporated into a composition of the present invention to maintain hydration or rehydrate the skin. Moisturizers that prevent water from evaporating from the skin by providing a protective coating are called emollients. Additionally an emollient provides a softening or soothing effect on the skin surface and is generally considered safe for topical use. Preferred emollients include mineral oils, lanolin, petrolatum, capric/caprylic triglyceraldehydes, cholesterol, silicones such as dimeticone, cyclometicone, almond oil, jojoba oil, avocado oil, castor oil, sesame oil, sunflower oil, coconut oil and grape seed oil, cocoa butter, olive oil aloe extracts, fatty acids such as oleic and stearic, fatty alcohols such as cetyl and hexadecyl (ENJAY), diisopropyl adipate, hydroxybenzoate esters, benzoic acid esters of C₉₋₁₅-alcohols, isononyl iso-nonanoate, ethers such as polyoxypropylene butyl ethers and polyoxypropylene cetyl ethers, and C₁₂₋₁₅-alkyl benzoates, and mixtures thereof. The most preferred emollients are hydroxybenzoate esters, aloe vera, C₁₂₋₁₅-alkyl benzoates, and mixtures thereof. An emollient is present in an amount of about 1 wt. % to about 20 wt. % of the total weight of the composition. The preferred amount of emollient is about 2 wt. % to about 15 wt. %, and most preferably about 4 wt. % to about 10 wt. %.

Moisturizers that bind water, thereby retaining it on the skin surface are called humectants. Suitable humectants can be incorporated into a composition of the present invention such as glycerin, polypropylene glycol, polyethylene glycol, lactic acid, pyrrolidon carboxylic acid, urea, phopholipids, collagen, elastin, ceramides, lecithin sorbitol, PEG-4, and mixtures thereof. Additional suitable moisturizers are polymeric moisturizers of the family of water soluble and/or swellable/and/or with water gelating polysaccarides such as hyaluronic acid, chitosan and/or a fucose rich polysaccharide which is e.g. available as Fucogel®1000 (CAS-Nr. 178463-23-5) by SOLABIA S. One or more humectants are optionally present at about 0.5 wt. % to about 8 wt. % in a composition of the present invention, preferably about 1 wt. % to about 5 wt. %.

The aqueous phase of the compositions of the present invention can contain the usual cosmetic additives such as alcohols, especially lower alcohols, preferably ethanol and/or isopropanol, low diols oder polyols and their ethers, preferably -propylenglycol, glycerine, ethyleneglycol, ethyleneglycol monoethyl- or monobutylether, propyleneglycol monomethyl- or -monoethyl- or -monobutylether, diethyleneglycol monomethyl-or monoethylether and analogue products, polymers, foam stabilisators; electrolytes and especially one or more thickeners. Thickeners that may be used in formulations of the present invention to assist in making the consistency of a product suitable include carbomer, siliciumdioxide, magnesium and/or aluminum silicates, beewax, stearic acid, stearyl alcohol polysaccharides and their derivatives such as xanthan gum, hydroxypropyl cellulose, polyacrylamides, acrylate crosspolymers preferably a carbomer, such as carbopole® of type 980, 981, 1382, 2984, 5984 alone or mixtures thereof. Suitable neutralizing agents which may be included in the composition of the present invention to neutralize components such as e.g. an emulsifier or a foam builder/stabilizer include but are not limited to alkali hydroxides such as a sodium and potassium hydroxide; organic bases such as diethanolamine (DEA), triethanolamine (TEA), aminomethyl propanol, and mixtures thereof; amino acids such as arginine and lysine and any combination of any foregoing. The neutralizing agent can be present in an amount of about 0.01 wt. % to about 8 wt. % in the composition of the present invention, preferably, 1 wt. % to about 5 wt. %.

The addition of electrolytes into the composition of the present invention may be necessary to change the behavior of a hydrophobic emulsifier. Thus, the emulsions/microemulsions of this invention may contain preferably electrolytes of one or several salts including anions such as chloride, sulfates, carbonate, borate and aluminate, without being limited thereto. Other suitable electrolytes can be on the basis of organic anions such as, but not limited to, lactate, acetate, benzoate, propionate, tartrate and citrate. As cations preferably ammonium, alkylammonium, alkali- or alkaline earth metals, magnesium-, iron- or zinc-ions are selected. Especially preferred salts are potassium and sodium chloride, magnesium sulfate, zinc sulfate and mixtures thereof. Electrolytes can be present in an amount of about 0.01 wt. % to about 8 wt. % in the composition of the present invention

The cosmetic compositions of the invention are useful as compositions for photoprotecting the human epidermis or hair against the damaging effect of ultraviolet irradiation, as sunscreen compositions. Such compositions can, in particular, be provided in the form of a lotion, a thickened lotion, a gel, a cream, a milk, an ointment, a powder or a solid tube stick and can be optionally be packaged as an aerosol and can be provided in the form of a mousse, foam or a spray. When the cosmetic composition according to the invention are provided for protecting the human epidermis against UV radiation or as sunscreen composition, they can be in the form of a suspension or dispersion in solvents or fatty substances, or alternatively in the form of an emulsion or microemulsion (in particular of O/W or W/O type, O/W/O or W/O/W-type), such as a cream or a milk, a vesicular dispersion, in the form of an ointment, a gel, a solid tube stick or an aerosol mousse. The emulsions can also contain anionic, nonionic, cationic or amphoteric surfactants.

The following examples are provided to further illustrate the processes and compositions of the present invention. These examples are illustrative only and are not intended to limit the scope of the invention in any way.

### EXAMPLE 1

### Improvement in Photostability of Encapsulated Cinnamate Derivatives in a Topical Sunscreen Composition by the Addition of (Non-Encapsulated) UV A and/or UV B and/or Broad Spectrum Screening Agents

An emulsion as shown in Table 1 was used. Into this emulsion either 5% of 2-ethylhexyl-4-methoxycinnamate (EHMC) as reference or a corresponding amount of encapsulated EHMC (Eusolex® UV-pearls™ OMC or similar microencapsulated EHMC) was incorporated and an additional amount or a combination of additional non-encapsulated UV-A, UV-B or broad spectrum sunscreens as indicated in Table 2. As is known to those skilled in the art lipophilic UV screens such as EHMC are added to the oil phase and hydrophilic UV-screens are added to the water phase. At which step the ‘encapsulated cinnamate’ is added to the emulsion is not critical. It can e.g. be added to the water phase or can be incorporated into the finished formulation before or after homogenization.

The photostability of the emulsions was determined according to G. Berset & H. Gonzenbach (COLIPA Task force); Proposed protocol for determination of photostability. Part I: cosmetic UV-filters, Int. J. Cosmet. Sci. 18, 167-177 (1996).

The decrease in absorption to 70% during irradiation of the reference is due to E/Z isomerization of commercial E-EHMC based on their different extinction coefficient (E-EHMC: ε=24'300, Z-EHMC: ε=14'500).

Encapsulated cinnamate derivatives may be prepared by means of various effective encapsulation methods such as e.g. the sol-gel method: WO 00/72806 (sunscreen composition containing sol-gel microcapsules), interfacial polymerization method: DE 2722973 (Encapsulation of products by interfacial polymerization), coacervation method: WO 9822210 (Chitin or chitin derivative microcapsules containing a hydrophobic sunscreen) or solvent evaporation method: European Polymer Journal (2001), 37(5), 955-963 (Microencapsulation by solvent evaporation), J. Controlled release 13, 33-41 (Microspheres of hyaluronic acid esters), a emulsion/interfacial emulsion polymerization or a method as described in EP 0934773 (Microcapsules having specific wall and method for producing the same) and in Fragrance Journal 2002, (30)7, 62-67 (New ingredients for UV protection. The caracteristic and application of microcapsule involving UV absorber).

TABLE 1
O/W sunscreen emulsion

| | | % |
|---|---|---|
| A) | Glyceryl Myristate | 3.00 |
| | Cetyl Alcohol | 1.00 |
| | UV-filter | |
| | Silicone 200/350 cs | 2.00 |
| | Tegosoft TN (=Finsolv) | 14.00 |
| | Amphisol A | 2.00 |
| | BHT | 0.05 |
| B) | Edeta BD | 0.10 |
| | Phenonip | 0.60 |
| | Water | ad 100 |
| | Propylene Glycol | 5.00 |
| | Carbopol ETD 2001 | 0.30 |
| | Tris 25% sol. | 3.8 |
| C) | encapsulated cinnamate | |

Procedure:
Heat part A) and B) to 85° C. while stirring. Add the additional non-encapsulated UV-A and/or UV-B and/or broad spectrum screen in the desired concentrations, based on their solubility, to the water or the oil phase. When homogeneous, add part B) to A) under agitation. Cool to about 45° C. while stirring Then add part C). Cool to ambient temperature while stirring. Homogenize again to achieve a small particle size.

TABLE 2
Photostability results

| Sample | Absorption |
|---|---|
| Reference containing 5% of free | 70% |
| 2-ethylhexyl-4-methoxy cinnamate | |
| Eusolex ® UV-pearls ™ OMC | 35% |
| Eusolex ® UV-pearls ™ OMC + | 48% |
| 2% avobenzone | |
| Eusolex ® UV-pearls ™ OMC + | 70% |
| 2% avobenzone + 1.8% octocrylene | |
| Eusolex ® UV-pearls ™ OMC + | 75% |
| 4% 4-methylbenzylidene camphor | |
| EHMC encapsulated via interfacial polymerization | 31% |
| EHMC encapsulated via interfacial polymerization + | 45% |
| 2% avobenzone | |
| EHMC encapsulated via interfacial polymerization + | 73% |
| 2% avobenzone + 1.8% octocrylene | |
| EHMC encapsulated in a silicon-based network polymer | 32% |
| EHMC encapsulated in a silicon-based network polymer + | 60% |
| 4% Parsol 5000 | |
| EHMC encapsulated in a silicon-based network polymer + | 64% |
| 4% TiO₂ AS | |

As is evident from the results above, there is a significant decrease of photostability by using encapsulated EHMC compared to ‘free EHMC’ in an emulsion. However, adding an additional non-encapsulated UV-A and/or UV-B and/or broad spectrum screen enhances the photostability and it is possible to obtain compositions which are as stable as the reference.

It should be understood that the foregoing description is only illustrative of the present invention. Various alternatives and modifications can be devised by those skilled in the art without departing from the invention. Accordingly, the present invention is intended to embrace all such alternatives, modifications and variances which fall within the scope of the appended claims.

EXAMPLE 2
O/W sun milk with pigments

| Ingredients | | INCI Nomenclature | % w/ [symbol] |
|---|---|---|---|
| A) | PARSOL SLX | Polysilicone-15 | 6.00 |
| | Neo Heliopan AP | | 3.00 |
| | Tinosorb S | Hydrogenated Cocoglycerides | 3.00 |
| | Lanette O | Cetearyl Alcohol | 2.00 |
| | Myritol 318 | Caprylic/capric Triglyceride | 6.00 |
| | Mineral oil | Mineral oil | 2.00 |
| | Vitamin E acetate | Tocopheryl Acetate | 1.00 |
| | Prisorine 3515 | Isostearyl Alcohol | 4.00 |
| B) | Edeta BD | Disodium EDTA | 0.10 |
| | Phenonip | Phenoxyethanol & Methylparaben & | 0.60 |
| | | Ethylparaben & Propylparaben & | |
| | | Butylparaben | |
| | Amphisol K | Potassium Cetyl Phosphate | 2.00 |
| | Water deionized | Aqua | ad 100 |
| | 1,2-Propylen Glycol | Propylene Glycol | 5.00 |
| | Carbopol 981 | Carbomer | 0.30 |
| | Tinosorb M | Methylene Bis-Benzotriazolyl | 6.00 |
| | | Tetramethylbutylphenol | |
| | KOH 10% solution | Potassium Hydroxyde | 2.10 |
| C) | ‘encapsulated | | 10-50 |
| | cinnamate’ | | |

Procedure:
Heat part A)and B) to 85° C. while stirring. When homogeneous, add part B) to A) under agitation. Cool to ambient temperature while stirring and add part C). Homogenize to achieve a small particle size.

EXAMPLE 3
Sun milk waterproofed

| Ingredients | | INCI Nomenclature | % w/ [symbol] |
|---|---|---|---|
| A) | PARSOL SLX | Polysilicone-15 | 6.00 |
| | PARSOL 1789 | Butyl Methoxydibenzoylmethane | 2.00 |
| | PARSOL 5000 | 4-Methylbenzylidene Camphor | 4.00 |
| | Uvinul T 150 | Ethylhexyltriazone | 2.00 |
| | Silicone DC | Dimethicone | 1.00 |
| | 200/350 cs | | |
| | Lanette O | Cetearyl Alcohol | 2.00 |
| | Softisan 100 | Hydrogenated Coco-Glycerides | 3.00 |
| | Tegosoft TN | C12-15 Alkyl Benzoate | 6.00 |
| | Cetiol B | Dibutyl Adipate | 7.00 |
| | Vitamin E acetate | Tocopheryl Acetate | 2.00 |
| | BHT | BHT | 0.05 |
| | Edeta BD | Disodium EDTA | 0.10 |
| | Phenonip | Phenoxyethanol & Methylparaben & | 0.60 |
| | | Ethylparaben & Propylparaben & | |
| | | Butylparaben | |
| | Amphisol | Cetyl Phosphate DEA | 2.00 |
| B) | Water deionized | Aqua | ad |
| | Propylene Glycol | Propylene Glycol | 5.00 |
| | Carbopol 980 | Carbomer | 0.30 |
| | KOH (10% sol.) | Potassium Hydroxide | 1.50 |
| C) | ‘encapsulated | | 10-50 [symbol] |
| | cinnamate’ | | |

Procedure:
Heat part A)and B) to 85° C. while stirring. When homogeneous, add part B) to A) under agitation. Cool to ambient temperature while stirring and add part C). Homogenize to achieve a small particle size.

EXAMPLE 4
Sun milk for babies and children

| Ingredients | | INCI Nomenclature | % w/ [symbol] |
|---|---|---|---|
| A) | Titanium Dioxide | Titanium Dioxide microfine | 4.00 |
| | Tegosoft TN | C12-15 Alkyl Benzoate | 5.00 |
| | Silicone 2503 | Stearyl Dimethicone | 2.00 |
| | Cosmetic | | |
| | Wax | | |
| | Cetyl Alcohol | Cetyl Alcohol | 1.00 |
| | Butylated | BHT | 0.05 |
| | Hydroxytoluene | | |
| | Estol GMM 3650 | Glyceryl Myristate | 4.00 |
| | Edeta BD | Disodium EDTA | 0.10 |
| | Phenonip | Phenoxyethanol & Methylparaben & | 0.60 |
| | | Ethylparaben & Propylparaben & | |
| | | Butylparaben | |
| | Amphisol A | Cetyl Phosphate | 2.00 |
| B) | Water deionized | Aqua | ad 10 [symbol] |
| | Carbopol 980 | Carbomer | 0.6 |
| | Glycerine | Glycerine | 3.00 |
| | KOH sol. 10% | Potassium Hydroxide | 2.4 |
| C) | ‘encapsulated | | 10-50 |
| | cinnamate’ | | |

Procedure:
Heat part A)and B) to 85° C. while stirring. When homogeneous, add part B) to A) under agitation. Cool to ambient temperature while stirring and add part C). Homogenize to achieve a small particle size.

EXAMPLE 5
High protective sun milk

| Ingredients | | INCI Nomenclature | % w/ [symbol] |
|---|---|---|---|
| A) | PARSOL SLX | Polysilicone-15 | 6.00 |
| | PARSOL 1789 | Butyl Methoxydibenzoylmethane | 2.00 |
| | PARSOL 5000 | 4-Methylbenzylidene Camphor | 4.00 |
| | Uvinul T 150 | | 2.00 |
| | Silicone DC | Dimethicone | 1.00 |
| | 200/350 cs | | |
| | Lanette O | Cetearyl Alcohol | 2.00 |
| | Softisan 100 | Hydrogenated Coco-Glycerides | 3.00 |
| | Tegosoft TN | C12-15 Alkyl Benzoate | 6.00 |
| | Cetiol B | Dibutyl Adipate | 7.00 |
| | Vitamin E acetate | Tocopheryl Acetate | 2.00 |
| | BHT | BHT | 0.05 |
| | Edeta BD | Disodium EDTA | 0.10 |
| | Phenonip | Phenoxyethanol & Methylparaben & | 0.60 |
| | | Ethylparaben & Propylparaben & | |
| | | Butylparaben | |
| | Amphisol K | Potassium Cetyl Phosphate | 2.00 |
| | Water deionized | Aqua | ad 10 [symbol] |
| | Propylene Glycol | Propylene Glycol | 5.00 |
| | Carbopol 980 | Carbomer | 0.30 |
| | KOH (10% sol.) | Potassium Hydroxide | 1.50 |
| C) | ‘encapsulated | | 10-50% |
| | cinnamate’ | | |
| D) | Perfume | Perfume | q.s. |

Procedure:
Heat part A)and B) to 85° C. while stirring. When homogeneous, add part B) to A) under agitation. Cool to ambient temperature while stirring and add part C) and D). Homogenize to achieve a small particle size.

EXAMPLE 6
Water-free sun gel

| Ingredients | | INCI Nomenclature | % w/w |
|---|---|---|---|
| A) | PARSOL MCX | Ethylhexyl Methoxycinnamate | 6.00 |
| | PARSOL 1789 | Butyl Methoxydibenzoylmethane | 4.00 |
| | PARSOL 5000 | 4-Methylbenzylidene Camphor | 4.00 |
| | Uvasorb HEB | Diethylhexyl Butamido Triazone | 1.50 |
| | Uvinul A plus | | 2.00 |
| | Vitamin E acetate | Tocopheryl Acetate | 1.50 |
| | Tegosoft TN | C12-15 Alkyl Benzoate | 9.00 |
| | Elefac I-205 | Ethylhexyldodecyl Neopentanoate | 2.00 |
| | Alcohol | Alcohol | ad 100 |
| | Isopropyl Alcohol | Isopropyl Alcohol | 20.00 |
| B) | Klucel MF | Hydroxypropylcellulose | 2.00 |
| C) | ‘encapsulated | | 10-50 |
| | cinnamate’ | | |
| D) | perfume | | q.s. |

Procedure:
Mix part A) and B) while stirring. When homogeneous, add part C) and D) under agitation.

EXAMPLE 7
Sun gel

| Ingredients | | INCI Nomenclature | % w/ [symbol] |
|---|---|---|---|
| A) | Pemulen TR-2 | Acrylates/C10-30 Alky Acrylate | 0.60 |
| | | Crosspolymer | |
| | Phenonip | Phenoxyethanol & Methylparaben & | 0.60 |
| | | Ethylparaben & Propylparaben & | |
| | | Butylparaben | |
| | Edeta BD | Disodium EDTA | 0.1 |
| | Aqua | Aqua | ad 100 |
| B) | PARSOL 1789 | Butyl Methoxydibenzoylmethane | 4.00 |
| | PARSOL 340 | Octocrylene | 3.00 |
| | Tegosoft TN | C12-15 Alkyl Benzoate | 15.00 |
| | Antaron V-216 | PVP/Hexadecene Copolymer | 1.00 |
| | Vitamin E acetate | Tocopheryl Acetate | 0.50 |
| | Uvinul TiO2 | Titanium Dioxide | 5.00 |
| | Butylated | BHT | 0.05 |
| | Hydroxytoluene | | |
| | Cremophor RH 410 | PEG-40 Hydrogenated Castor Oil | 0.50 |
| | TrisAmino | Tromethamine | 0.50 |
| C) | ‘encapsulated | | 10-50% |
| | cinnamate’ | | |
| D) | Perfume | Perfume | q.s. |

Procedure:
Heat part A)and B) to 85° C. while stirring. When homogeneous, add part B) to A) under agitation. Cool to ambient temperature while stirring and add part C) and D). Homogenize to achieve a small particle size.

EXAMPLE 8
High protection WO sun milk

| Ingredients | | INCI Nomenclature | % w/ [symbol] |
|---|---|---|---|
| A) | PARSOL 1789 | Butyl Methoxydibenzoylmethane | 2.00 |
| | PARSOL 5000 | 4-Methylbenzylidene Camphor | 4.00 |
| | Uvinul T 150 | Ethylhexyl Triazone | 2.00 |
| | Uvinul TiO2 | Titanium Dioxide and | 5.00 |
| | | Trimethoxycaprylylsilane | |
| | Arlacel P 135 | PEG-30 Dipolyhydroxystearate | 2.00 |
| | Tegosoft TN | C12-15 Alkyl Benzoate | 5.00 |
| | Cosmacol EMI | Di-C12-13 Alkyl Malate | 6.00 |
| | Miglyol 840 | Propylene Glycol | 6.00 |
| | | Dicaprylate/Dicaprate | |
| | Butylated | BHT | 0.05 |
| | Hydroxytoluene | | |
| | Phenonip | Phenoxyethanol & Methylparaben & | 0.60 |
| | | Ethylparaben & Propylparaben & | |
| | | Butylparaben | |
| B) | Deionized water | Aqua | ad 10 [symbol] |
| | Glycerin | Glycerin | 5.00 |
| | Edeta | Disodium EDTA | 0.1 |
| | NaCl | Sodium Chloride | 0.30 |
| C) | PARSOL HS | Phenylbenzyimidazole | 4.00 |
| | | Sulphonic Acid | |
| | Water | Aqua | 20.00 |
| | Triethanolamine | Triethanolamine | 2.50 |
| | 99%. | | |
| D) | ‘encapsulated | | 10-50 |
| | cinnamate’ | | |
| E) | Perfume | | q.s. |

Procedure:
Heat part A), B) and C) to 85° C. while stirring. When homogeneous, add part B) and C) to A) under agitation. Cool to ambient temperature while stirring and add part D) and E). Homogenize to achieve a small particle size.

EXAMPLE 9
W/O milk with pigments

| Ingredients | | INCI Nomenclature | % w/ [symbol] |
|---|---|---|---|
| A) | Cremophor WO 7 | PEG-7 Hydrogenated Castor Oil | 6.00 |
| | Elfacos ST 9 | PEG-45/Dodecyl Glycol Copolymer | 2.00 |
| | PARSOL 1789 | Butyl Methoxydibenzoylmethane | 3.00 |
| | Tinosorb S | | 5.00 |
| | PARSOL 5000 | 4-Methylbenzylidene Camphor | 4.00 |
| | Uvinul TiO2 | Titanium Dioxide | 2.00 |
| | microfine ZnO | Zinc Oxide | 2.00 |
| | Microcrystalline | Microcrystalline Wax | 2.00 |
| | wax | | |
| | Miglyol 812 | Caprylic/capric Triglyceride | 5.00 |
| | Vitamin E acetate | Tocopheryl Acetate | 1.00 |
| | Jojoba oil | Simmondsia Chinensis Seed Oil | 5.00 |
| | Edeta BD | Disodium EDTA | 0.10 |
| | Butylated | BHT | 0.05 |
| | Hydroxytoluene | | |
| | Phenonip | Phenoxyethanol & Methylparaben & | 0.60 |
| | | Ethylparaben & Propylparaben & | |
| | | Butylparaben | |
| B) | Water deionized | Aqua | ad [symbol] |
| | Glycerin | Glycerin | 5.00 |
| C) | Neo Heliopan AP | | 2.00 |
| | Water deionized | Aqua | 20.00 |
| | KOH 10% solution | Potassium Hydroxide | 4.00 |
| D) | ‘encapsulated | | 10-50 [symbol] |
| | cinnamate’ | | |
| E) | Perfume | Perfume | q.s. |

Procedure:
Heat part A), B) and C) to 85° C. while stirring. When homogeneous, add part B) and C) to A) under agitation. Cool to ambient temperature while stirring and add part D) and E). Homogenize to achieve a small particle size.

EXAMPLE 10
Protective Day cream with Vitamin C

| | | | % |
|---|---|---|---|
| | Ingredients | INCI Nomenclature | w/w |
| A) | PARSOL SLX | Polysilicone-15 | 4.00 |
| | PARSOL 1789 | Butyl | 1.50 |
| | | Methoxydibenzoylmethane | |
| | Glyceryl Myristate | Glyceryl Myristate | 2.00 |
| | Cetyl Alcohol | Cetyl Alcohol | 0.50 |
| | Myritol 318 | Caprylic/Capric Triglyceride | 5.00 |
| | Crodamol DA | Diisopropyl Adipate | 5.00 |
| | Viatmin E acetate | Tocopheryl Acetate | 2.00 |
| | Butylated | BHT | 0.05 |
| | Hydroxytoluene | | |
| | Phenonip | Phenoxyethanol & | 0.60 |
| | | Methylparaben & | |
| | | Ethylparaben & | |
| | | Propylparaben & | |
| | | Butylparaben | |
| | Edeta BD | Disodium EDTA | 0.10 |
| | Amphisol K | Potassium Cetyl Phosphate | 2.00 |
| B) | Water deionized | Aqua | ad: |
| | 1,2-Propylene Glycol | Propylene Glycol | 2.00 |
| | D-Panthenol 75 L | Panthenol | 2.00 |
| | Ethanol | Ethanol | 5.00 |
| | Allantoin | Allantoin | 0.20 |
| | Carbopol ETD 2001 | Carbomer | 0.30 |
| | KOH 10% sol. | Potassium Hydroxide | 1.50 |
| C) | Water | Aqua | 10.00 |
| | Stay-C 50 | Sodium Ascorbyl Phosphate | 0.50 |
| D) | ‘encapsulated cinnamate’ | | 10-50% |
| E) | Perfume | Perfume | q.s. |

Procedure:
Heat part A), B) and C) to 85° C. while stirring. When homogeneous, add part B) and C) to A) under agitation. Cool to ambient temperature while stirring and add part D) and E). Homogenize to achieve a small particle size.

1. A method of enhancing the photostability of an encapsulated cinnamate derivative in a topical sunscreen composition which comprises introducing into such sunscreen composition an effective amount of at least one additional non-encapsulated sunscreen.

2. The method as in claim 1 wherein the additional non-encapsulated sunscreen is a UV-B or a broad spectrum or a combination of a UV-B and/or a broad spectrum and a UV-A sunscreen.

3. The method as in claim 1 wherein the encapsulated cinnamate is one prepared by the sol-gel method, a solvent evaporation method, a coacervation method, an interfacial polymerization method or an emulsion/interfacial emulsion polymerization method.

4. The method as in claim 1 wherein the microcapsules have as a core a cinnamic acid derivative which is surrounded by a shell of silicon based polymer such as ‘sol-gel glass’, a silicon-based network polymer, a ‘silicone-resin polypeptide’, a polyurea, a polyurethane, a polyamide, a polyester or a combination thereof.

5. The method as in claim 3 wherein the encapsulating cinnamate is prepared by the sol-gel method.

6. The method as in claim 1 wherein the cinnamate is of the formula I 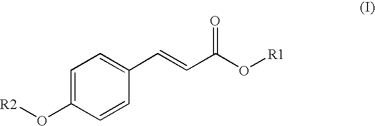
wherein R¹, R² are, independently, hydrogen or saturated straight or branched chain alkyl containing 1 to 21, preferably 1 to 8 carbon atoms, such as methyl, ethyl, propyl, isopropyl, butyl, sec. butyl, isobutyl, pentyl, neopentyl, hexyl, 2-ethyl-hexyl, and octyl.

7. The method as in claim 5 wherein the cinnamate is 2-ethylhexyl-p-methoxycinnamate

8. The method as in claim 1 wherein the additional non-encapsulated sunscreen is selected from DEA-Methoxycinnamate, diethylhexyl butamido triazine, diisopropyl methyl cinnamate, 1-(3,4-dimethoxyphenyl)-4,4-dimethyl-1,3-pentanedione, drometrizole trisiloxane, benzophenone-3, benzophenone-4,3-benzylidene camphor, benzylidene camphor sulfonic acid, bis-ethylhexyloxyphenol methoxyphenyl triazine, butyl methoxydibenzoylmethane, camphor benzalkonium methosulfate, ethyl diisopropylcinnamate, 2-ethylhexyl dimethoxybenzylidene dioxoimidazolidine propionate, ethylhexyl dimethyl PABA, ethylhexyl salicylate, ethylhexyl triazone, ethyl PABA, homosalate, isoamyl p-methoxycinnamate, menthyl anthranilate, 4-methylbenzylidene camphor, methylene-bis-benzotriazolyl tetramethylbutylphenol, octocrylene, PABA, phenylbenzimidazole sulfonic acid, polyacrylamidometyl benzylidene camphor, polysilicone-15, potassium phenylbenzimidazole sulfonate, sodium phenylbenzimidazole sulfonate, TEA-salicylate, terephthalidene dicamphor sulfonic acid, 2,2-(1,4-phenylene) bis-(1H-benzimidazol-4,6-disulfonic acid, 2-(4-Diethylamino-2-hydroxy-benzoyl)-benzoic acid hexylester, microfine titanium dioxide and microfine zinc oxide.
